(19) United States
(12) Patent Application Publication
(10) Pub. No.: US 2017/0000115 A1
(43) Pub. Date: Jan. 5, 2017

(54) Antimicrobial Substrates And Methods Of Use Thereof

(71) Applicant: XMICROBIAL, LLC, Scituate, MA (US)

(72) Inventors: Roger A. Nassar, Swampscott, MA (US); Shirley Young, Scituate, MA (US); Marion L. Chiattello, Cedar Falls, IA (US); Mark Oman, West Des Moines, IA (US)

(21) Appl. No.: 15/106,626

(22) PCT Filed: Dec. 22, 2014
(86) PCT No.: PCT/US2014/071863

§ 371 Date: Jun. 20, 2016

Related U.S. Application Data
(60) Provisional application No. 61/920,322, filed on Dec. 23, 2013.

(51) Int. Cl. A01N 33/12; A01N 25/10; C08G 65/00; A61L 2/23

(52) U.S. Cl. CPC A01N 33/12; A61L 2/23; A01N 25/10; C08G 65/00

(57) ABSTRACT

Provided is an antimicrobial substrate comprising a substrate to which is covalently bonded an antimicrobial polymer, in which the antimicrobial polymer comprises an alkylene oxide backbone to which are attached one or more alkyl and/or alkylene oxide primary and/or secondary branches, at least one of the alkyl or alkylene oxide primary and/or secondary branches is functionalized with a quaternary ammonium or a fluorinated group, or at least two of the alkyl and/or alkylene oxide primary and/or secondary branches are functionalized with a quaternary ammonium and a fluorinated group. The antimicrobial substrate can be used, for example, in a method for protecting an object against microbial infection, microbial colonization, or microbial transinfection comprising providing to the object an antimicrobial substrate.

### FIELD

The present disclosure is directed, in part, to antimicrobial substrates and methods of using the same.

### BACKGROUND

Pathogenic microbes such as bacteria, virus, fungi, yeast, and algae, are known to cause serious illnesses and death. Microbes can be found in a variety of places and objects such as hospitals, gloves, aprons, shields, implants, kitchen counters, computer keyboards, telephones, factories, animals, food processing equipment, pharmaceuticals, air, water, and farming equipment. People, animals, birds, and objects that come into contact with such microbes are at risk of being infected or contaminated by these microbes. There is, therefore, a need to kill or inactivate the microbes or prevent the surfaces from being invaded by the microbes.

Proposals have been made to modify the surfaces of materials with antimicrobial agents so that microbes coming into contact with such surfaces would be inactivated or killed. For example, antimicrobials have been proposed to be attached to various materials through non-covalent bonding such as coating or painting. Examples of articles whose surfaces have been non-covalently modified by antimicrobials can be found in U.S. Patent Application Publication Nos. 2013/0110237 A1 and 2010/0136072 A1, where the surface has been coated with antimicrobials. However, such non-covalently modified surfaces have certain limitations; for example, the antibacterial coating tends to leach away with time, thus, reducing the useful life of the coated surface. In addition, the antimicrobial could contaminate the product that it comes contact with. Moreover, coatings that work by the mechanism of leaching into a solution to kill the microbe will not work with airborne microbes.

Covalently bonded surfaces are advantageous since they are more permanent and less contaminating, and may also be suitable for use with airborne microbes. Such surfaces also have been proposed. For example, WO 2002/085542 A1 discloses surfaces comprised of amphipathic compounds such as quaternized poly(N-alkyl vinylpyridine) or poly(N-alkyl ethyleneimine) polymers covalently bonded to a glass surface reportedly for the prevention of accumulation of microorganisms wherein such accumulation has a deleterious effect on human or animal health. WO 2008/000429 A1 discloses articles reportedly exhibiting antimicrobial efficacy, which articles contain a carrier, a spacer attached to the carrier, for example, a polymer, and one or more quaternary ammonium groups attached directly or indirectly to the spacer, said articles reportedly for use in the manufacture of bottles, contact lenses, textiles, coatings, pellets, beads, and films.

Despite the above proposals, the majority of which are directed to the attachment of predominantly hydrophobic polymers to surfaces, there still exists an unmet need for surfaces that are covalently bonded to antimicrobials exhibiting tunable hydrophilic and fluorophilic chemical compositions.

### SUMMARY

One or more of the foregoing needs have been fulfilled by the present disclosure. Accordingly, the present disclosure provides an antimicrobial substrate comprising a substrate to which is covalently bonded an antimicrobial polymer, wherein said antimicrobial polymer comprises an alkylene oxide backbone to which are attached one or more alkyl and/or alkylene oxide primary branches, wherein at least one of the alkyl or alkylene oxide primary branches is functionalized with a quaternary ammonium group or a fluorinated group, or at least two of the alkyl and/or alkylene oxide primary branches are functionalized with a quaternary ammonium group and a fluorinated group, wherein at least one of the alkyl and/or alkylene oxide primary branches optionally contains one or more alkyl and/or alkylene oxide secondary branches that are functionalized with a quaternary ammonium group or a fluorinated group, or at least two of the alkyl and/or alkylene oxide secondary branches are functionalized with a quaternary ammonium group and a fluorinated group, and wherein said polymer is associated with an anion to maintain electro-neutrality when a quaternary ammonium group is present.

Also provided are methods of using an antimicrobial substrate as described herein. The inventive methods include a method for protecting an object against microbial infection, microbial colonization, or microbial transinfection comprising providing to the object an antimicrobial substrate.

Because the antimicrobial polymer is covalently bonded to the substrate, the polymer does not leach away over time, thereby improving the useful life of the antimicrobial substrate and reducing contamination of the object associated with the surface. Moreover, the covalently bonded antimicrobialpolymer is effective to kill or render inactive airborne microbes.

Additionally, because of the presence of two main groups having different chemical characteristics: a hydrophilic backbone (alkylene oxide) and hydrophobic and/or fluorophilic functionalities (ammonium and/or fluorinated groups), the overall performance and interaction of the surface coated with microbicidal polymer with its surrounding chemicals and surfaces can be tuned. For example, shorter alkylene in alkylene oxide groups and/or higher repeating number of alkylene oxide groups and/or shorter alkyl groups in ammoniums and/or shorter fluorinated groups lead to a more hydrophilic character in the microbicidal polymer. Such character generates stronger interactions between the antimicrobial substrate and surrounding hydrophilic entities. Conversely, longer alkylene in alkylene oxide groups and/or lower repeating number of alkylene oxide groups and/or longer alkyl groups in ammoniums and/or longer fluorinated groups all lead to a more hydrophobic character in the microbicidal polymer. Such character generates weaker interactions between the antimicrobial substrate and surrounding hydrophilic entities.

### BRIEF DESCRIPTION OF THE DRAWINGS

FIG. 1 depicts a method of preparing an antimicrobial substrate in accordance with an embodiment of the disclosure.

### DESCRIPTION OF EMBODIMENTS

The present disclosure provides antimicrobial substrates comprising a substrate to which is covalently bonded an antimicrobial polymer, wherein said antimicrobial polymer comprises an alkylene oxide backbone to which are attached one or more alkyl and/or alkylene oxide primary branches, wherein at least one of the alkyl or alkylene oxide primary branches is functionalized with a quaternary ammonium group or a fluorinated group, or at least two of the alkyl and/or alkylene oxide primary branches are functionalized with a quaternary ammonium group and a fluorinated group, wherein at least one of the alkyl and/or alkylene oxide primary branches optionally contains one or more alkyl and/or alkylene oxide secondary branches that are functionalized with a quaternary ammonium group or a fluorinated group, or at least two of the alkyl and/or alkylene oxide secondary branches are functionalized with a quaternary ammonium group and a fluorinated group, and wherein said polymer is associated with an anion to maintain electro-neutrality when a quaternary ammonium group is present.

As used herein, the term “antimicrobial” means microbicidal, i.e., having the ability to kill, repel, and/or inactivate a microorganism, as described herein.

The anion is any suitable negatively charged moiety that serves to neutralize the charge of a quaternary ammonium group, as described herein. The anion can be, for example, a halide (e.g., Cl⁻, F⁻, Br⁻, I⁻), an oxoanion (e.g., CO₃²⁻, HCO₃²⁻, OH⁻, NO³⁻, PO₄³⁻, or SO₄²⁻), or an organic anion (e.g., CH₃COO⁻, HCOO⁻, C₂O₄²⁻, or CN⁻).

The alkylene oxide backbone, alkyl or alkylene oxide primary branches, and alkyl or alkylene oxide secondary branches can have any number of suitable carbons, such as C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, or C₂₀. The number of carbons in the alkylene oxide backbone or alkyl or alkylene oxide primary or secondary branches is determined by the desired solubility properties and/or end use. For example, it will generally be understood that the larger the alkylene portion of the alkylene oxide backbone, primary branches, and/or secondary branches, and the longer the alkyl group in the primary branches and/or secondary branches, the less water or lower alcohol soluble the polymer will be. Inversely, the shorter the alkylene portion of the alkylene oxide backbone, primary branches, and/or secondary branches, and the shorter the alkyl group in the primary branches and/or secondary branches, the more water or lower alcohol soluble the polymer will be.

In some aspects, the alkylene oxide backbone comprises a propylene (C₃) oxide backbone, an ethylene (C₂) oxide backbone, or both propylene oxide and ethylene oxide units. The primary branch and/or secondary branch comprises at least one ethylene oxide unit or at least one propylene oxide unit.

In certain aspects, the alkylene oxide backbone is a propylene oxide of the formula:

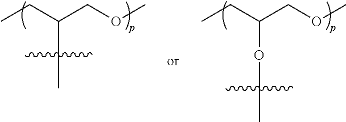

wherein p is 1 to 60. For example, p is a range of 1 to 50, 1 to 40, 1 to 30, 1 20, 2 to 20, 2 to 15, 2 to 12, 2 to 10, 2 to 8, 3 to 10, 3 to 8, 4 to 10, 4 to 8, 5 to 10, 5 to 8, 6 to 10, or 6 to 8; or p is 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 52, 54, 56, 58, or 60. The alkyl and/or alkylene oxide primary branches are attached to one or more of the three open sites shown above.

The alkylene oxide backbone can be attached to any suitable number of alkyl and/or alkylene oxide primary or secondary branches. The length of the alkyl branches and the number of primary or secondary branches is determined by the desired solubility properties and/or end use. In certain aspects, the alkylene oxide backbone comprises at least 2 (e.g., at least 3, at least 4, at least 4, at least 5, at least 6, at least 7, or at least 8) primary or secondary branches. The backbone can have an upper limit of any number of suitable branches, e.g., up to 100 (e.g., up to 80, up to 60, up to 40, up to 20, or up to 10) alkyl and/or alkylene oxide primary or secondary branches. These lower and upper limits with respect to the number of alkyl and/or alkylene oxide primary or secondary branches can be used in any combination (e.g., 2-100, 3-80, and 4-10, etc.). In certain aspects, the backbone has 2 to 20 primary branches. The alkylene oxide backbone is linked to 4 to 8 primary branches (e.g., ethylene oxide primary branches). Depending on the number and placement of branches, the polymer can be described as a star polymer, comb polymer, brush polymer, palm tree polymer, H-shaped polymer, or dumbbell polymer.

In some embodiments, the primary or secondary branch can be based on polyethylene glycol (PEG). For example, a branched PEG, e.g., an alkylene oxide backbone comprising 2 to 100 PEG branches, can be used as the antimicrobial polymer in accordance with an embodiment. In some embodiments, the polymer comprises 3 to 10 (e.g., 3, 4, 5, 6, 7, 8, 9, or 10) PEG branches.

The primary branch or the secondary branch, that is functionalized is of the formula

—(CH₂)ₙ—X,—(CH₂CH₂O)ₙ—X, or —(CH₂CH₂CH₂O)ₙ—X,

wherein

X is —(CH₂)ₘ—Y, when Y is a quaternary ammonium group as described herein or

X is —(CH₂)ₘ—NHC(O)—Y, when Y is a fluorinated group as described herein,

m is 0 to 10, and

n is 1 to 2,500.

The value of m determines the length of the alkylene linker (e.g., 0, 1, 2, 3, 4, 5, 6, 7, 8, 9, or 10). If no linker is necessary, then m is 0. In certain embodiments, m is 1 or 2.

The value of n determines, in part, the molecular weight of the antimicrobial polymer. The molecular weight of the polymer is as described herein, and n is a number that provides the desired molecular weight. Typically, n is 1 to 2,500 (e.g., 1 to 2,000, 2 to 1,000, 2 to 800, 2 to 600, 3 to 500, 3 to 400, 4 to 400, 4 to 300, etc.).

The antimicrobial polymer of the disclosure can be provided or prepared by any suitable method. For example, synthesis methods are described herein, and polymer starting materials can be prepared by methods known in the art or are commercially available (e.g., Sigma-Aldrich (St. Louis, Mo.), Dow Chemical (Midland, Mich.), JenKem Technology (Allen, Tex.)). Typical polymerization methods include ring opening polymerization, suspension polymerization, free radical polymerization, anionic polymerization, cationic polymerization, or metallocene catalysis and can include an initiator and/or a catalyst. Examples of a suitable catalyst include an acid catalyst, an alkali metal-based catalyst (e.g., NaOH, KOH, Na₂CO₃), a metal oxide catalyst, an Mg-based catalyst, a Ca-based catalyst, an Al-based catalyst, and a combination thereof.

The antimicrobial polymer can be any suitable average molecular weight and usually is a function of the ratio of starting materials and synthesis method. Typically, the molecular weight is tuned based on the desired solubility properties and/or end use. For example, the number, weight, or volume average molecular weight can be at least about 200 g/mol (e.g., at least about 300 g/mol, at least about 500 g/mol, at least about 800 g/mol, at least about 1,000 g/mol, at least about 1,500 g/mol, at least about 2,000 g/mol) and/or up to about 100,000 g/mol (e.g., up to about 90,000 g/mol, up to about 80,000 g/mol, up to about 70,000 g/mol, up to about 60,000 g/mol, up to about 50,000 g/mol, up to about 40,000 g/mol, up to about 30,000 g/mol, up to about 20,000 g/mol, or up to about 10,000 g/mol). These lower and upper limits with respect to the number, weight, or volume average molecular weight can be used in any combination to describe the polymer molecular weight range (e.g., about 200 to about 100,000 g/mol, about 300 g/mol to about 50,000 g/mol, and about 1,000 to about 20,000 g/mol, etc.).

The antimicrobial polymer can be characterized quantitatively using known methods. For example, molecular weight determinations can be made using gel permeation chromatography (also known as size exclusion chromatography and gel filtration chromatography), nuclear magnetic resonance spectroscopy (NMR), matrix-assisted laser desorption/ionization mass spectroscopy (MALDI), light scattering (e.g., low angle and multi angle), small angle neutron scattering (SANS), sedimentation velocity, end group analysis, osmometry, cryoscopy/ebulliometry, and viscometry.

In some aspects, at least one of the alkyl and/or alkylene oxide primary branches is functionalized with a quaternary ammonium group (such as substituent Y described above), which can have the formula —N⁺R¹R²R³. Substituents R¹, R², and R³ are independently selected from alkyl, alkenyl, cycloalkyl, and aryl. In some embodiments, R¹, R², and R³ are selected based on the desired properties and/or end use. For example, it will generally be understood that the larger the number of carbons in R¹, R², and/or R³, the more hydrophobic the final polymer-coated surface will be. Inversely, the fewer the number of carbons in R¹, R², and/or R³, the more hydrophilic the final polymer-coated surface will be. In some embodiments, R¹, R², and R³ are independently alkyl, such as a C₁₋₂₀ alkyl (e.g., C₁₋₁₈ alkyl, C₁₋₁₆ alkyl, C₁₋₁₄ alkyl, C₁₋₁₂ alkyl, or C₁₋₁₀ alkyl). In specific examples, R¹ and R² are each a lower alkyl (e.g., methyl, ethyl, propyl, butyl, or pentyl) and R³ is an alkyl selected from hexyl, octyl, decyl, dodecyl, tetradecyl, hexadecyl, and octadecyl. In some embodiments, R¹ and R² are methyl, and R³ is decyl, dodecyl, or octadecyl.

In some aspects, at least one of the alkyl and/or alkylene oxide primary branches is functionalized with a fluorinated group (such as substituent Y described above). The fluorinated group can be, for example, fluoroalkyl, fluoroalkenyl, fluorocycloalkyl, or fluoroaryl), a perfluorinated group such as perfluoroalky, perfluoralkenyl, perfluorcycloalkyl, or perfluoroaryl. In an embodiment, the perfluorinated group is perfluoroalkyl, such as C₁₋₁₈ perfluoroalkyl (e.g., C₁₋₁₆ perfluoroalkyl, C₁₋₁₄ perfluoroalkyl, C₁₋₁₂ perfluoroalkyl, or C₁₋₁₀ perfluoroalkyl). In some embodiments, the fluoroalkyl is nonafluorobutyl and its isomers, heptafluoropropyl and its isomers, and pentafluoroethyl.

In certain other aspects, the antimicrobial polymer comprises at least two primary branches. One of the primary branches is functionalized with a quaternary ammonium group, and the other primary branch is functionalized with a fluorinated group. This type of hybrid polymer can have any number of quaternary ammonium or fluorinated groups (e.g., at least 1 of each, at least 2 of each, at least 3 of each, at least 4 of each, at least 5 of each, at least 10 of each, at least 15 of each, at least 20 of each, etc.). The polymer can comprise equal or unequal numbers of quaternary ammonium and fluorinated groups (e.g., 3 fluorinated groups and 1 quaternary ammonium group; or 4 fluorinated groups and 4 quaternary ammonium groups). The quaternary ammonium group and fluorinated group are as described herein.

As used herein, unless otherwise specified, the term “alkyl” means a saturated straight chain or branched non-cyclic hydrocarbon having an indicated number of carbon atoms (e.g., C₁-C₂₀, C₁-C₁₈, C₁-C₁₆, C₁-C₁₄, C₁-C₁₂, C₁-C₁₀, C₁-C₈, C₁-C₆, C₁-C₄, etc.). Representative saturated straight chain alkyls include methyl, ethyl, n-propyl, n-butyl, n-pentyl, n-hexyl, n-heptyl, n-octyl, n-nonyl, n-decyl, n-dodecyl, n-tetradecyl, n-hexadecyl, and n-octadecyl; while representative saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. An alkyl group can be unsubstituted or substituted.

As used herein, unless otherwise specified, the term “alkenyl group” means a straight chain or branched non-cyclic hydrocarbon having an indicated number of carbon atoms (e.g., C₂-C₂₀, C₂-C₁₈, C₂-C₁₆, C₂-C₁₄, C₂-C₁₂, C₂-C₁₀, etc.) and including at least one carbon-carbon double bond. Representative straight chain and branched alkenyls include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, and the like. Any unsaturated group (double bond) of an alkenyl can be unconjugated or conjugated to another unsaturated group. An alkenyl group can be unsubstituted or substituted.

The term “cycloalkyl,” as used herein, means a cyclic alkyl moiety containing from, for example, 3 to 7 carbon atoms, or from 5 to 6 carbon atoms. Examples of such moieties include cyclopropyl, cyclobutyl, cyclopentyl, cyclohexyl, and the like.

The term “aryl” refers to an unsubstituted or substituted aromatic carbocyclic moiety, as commonly understood in the art, and includes monocyclic and polycyclic aromatics such as, for example, phenyl, biphenyl, naphthyl, anthracenyl, pyrenyl, and the like. An aryl moiety generally contains from, for example, 6 to 30 carbon atoms, from 6 to 18 carbon atoms, from 6 to 14 carbon atoms, or from 6 to 10 carbon atoms. It is understood that the term aryl includes carbocyclic moieties that are planar and comprise 4n+2 π electrons, according to Hückel's Rule, wherein n=1, 2, or 3.

As used herein, unless otherwise specified, the term “substituted” means a group substituted by one or more substituents (e.g., 1, 2, 3, 4, 5, 6, etc.), such as, alkyl, alkenyl, alkynyl, cycloalkyl, aroyl, halo, haloalkyl (including trifluoromethyl), haloalkoxy (including trifluoromethoxy), hydroxy, alkoxy, cycloalkyloxy, heterocylooxy, oxo (═O), alkanoyl, aryl, arylalkyl, alkylaryl, heteroaryl, heteroarylalkyl, alkylheteroaryl, heterocyclo, aryloxy, alkanoyloxy, amino, alkylamino, arylamino, arylalkylamino, cycloalkylamino, heterocycloamino, alkanoylamino, aroylamino, aralkanoylamino, substituted alkanoylamino, substituted arylamino, substituted aralkanoylamino, thiol (mercapto), alkylthio, arylthio, arylalkylthio, cycloalkylthio, heterocyclothio, alkylthiono, arylthiono, arylalkylthiono, alkylsulfonyl, arylsulfonyl, arylalkylsulfonyl, sulfonamido (e.g., —SO₂NH₂), substituted sulfonamido, nitro, cyano, carboxy, carbamido, carbamyl (e.g., —CONH₂), substituted carbamyl (e.g., —CONH-alkyl, —CONH-aryl, —CONH-arylalkyl, or instances where there are two substituents on the nitrogen selected from alkyl or arylalkyl), alkoxycarbonyl, aryl, substituted aryl, substituted or unsubstituted heterocycloalkyl, and substituted or unsubstituted heteroaryl.

In some embodiments, the antimicrobial substrate is selected from

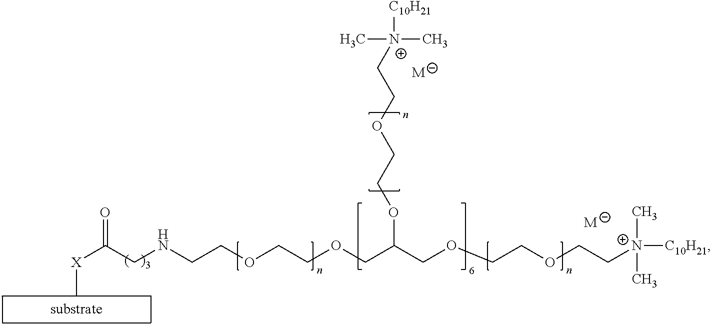

wherein:

X is O, S, or NH;

M⁻ is an anion; and

n is 2 to 2500.

In some aspects, X is O. In other aspects, X is NH. The anion M⁻ is any suitable negatively charged moiety that serves to neutralize the charge of a quaternary ammonium group, as described herein. The anion can be, for example, a halide (e.g., Cl⁻, F⁻, Br⁻, I⁻), an oxoanion (e.g., CO₃²⁻, HCO₃²⁻, OH⁻, NO³⁻, PO₄³⁻, or SO₄²⁻), or an organic anion (e.g., CH₃COO⁻, HCOO⁻, C₂O₄²⁻, or CN⁻). The substituent n is as described herein and determines, at least in part, the molecular weight of the polymer.

In some embodiments, the antimicrobial polymer has a polycation selected from polycation N,N-octadecyl, methyl-PEG; polycation N,N-dodecyl, methyl-PEG; polycation N,N-decyl, methyl-PEG; polycation N,N-octyl, methyl-PEG; polycation N,N-hexyl, methyl-PEG; and polycation N,N-hexyl, methyl-PEG.

Other examples of the antimicrobial polymer include: polycation N,N-octadecyl, methyl-PEG(440, ca. 2-arm); polycation N,N-dodecyl, methyl-PEG(440, ca. 2-arm); polycation N,N-octadecyl, methyl-PEG(2k, ca. 3-arm); polycation N,N-dodecyl, methyl-PEG(2k, ca. 3-arm); polycation N,N-octadecyl, methyl-PEG(3k, ca. 3-arm); polycation N,N-dodecyl, methyl-PEG(3k, ca. 3-arm); polycation N,N-decyl, methyl-PEG(10k, ca. 7-arm); polycation N,N-decyl, methyl-PEG(10k, ca. 3-arm); polycation N,N-decyl, methyl-PEG(20k, ca. 7-arm); polycation N,N-decyl, methyl-PEG(20k, ca. 3-arm); polycation N,N-decyl, methyl-PEG(40k, ca. 7-arm); polycation N,N-decyl, methyl-PEG(40k, ca. 3-arm); polycation N,N-octyl, methyl-PEG(10k, ca. 7-arm); polycation N,N-octyl, methyl-PEG(10k, ca. 3-arm); polycation N,N-octyl, methyl-PEG(20k, ca. 7-arm); polycation N,N-octyl, methyl-PEG(20k, ca. 3-arm); polycation N,N-octyl, methyl-PEG(40k, ca. 7-arm); polycation N,N-octyl, methyl-PEG(40k, ca. 3-arm); polycation N,N-hexyl, methyl-PEG(10k, ca. 7-arm); polycation N,N-hexyl, methyl-PEG(10k, ca. 3-arm); polycation N,N-hexyl, methyl-PEG(20k, ca. 7-arm); polycation N,N-hexyl, methyl-PEG(20k, ca. 3-arm); polycation N,N-hexyl, methyl-PEG(40k, ca. 7-arm); and polycation N,N-hexyl, methyl-PEG(40k, ca. 3-arm).

The disclosure further provides methods of using an antimicrobial substrate as described herein. Settings suitable for the use of the polymer described herein include, but not limited to, a home, office, hospital, clean room, research lab, veterinary settings, factory, construction site, public facility, dormitory, school, airport, stadium, park, playground, vehicles for air, land, and water, and agricultural environments.

In particular, the disclosure provides a method for protecting an object against microbial infection, microbial colonization, or microbial transinfection comprising providing to the object an antimicrobial substrate as described herein. The antimicrobial substrate can make up the entire object or comprise only a part of the object. In the presence of an antimicrobial substrate, it is believed that microbes that land on the antimicrobial substrate are either repelled or rendered inactive.

The antimicrobial substrate can be provided to the object by any suitable method, such as physical adhesion or chemical adhesion or introducing the antimicrobial substrate into the production process of the object. Examples of providing an antimicrobial substrate to an object include, e.g., spinning, coating, laminating, gluing, pasting, cementing, pressing, hot pressing, adhering, electrostatic adhering, binding, nailing, stapling, sewing, spraying, misting, rolling, brushing, drying, spray drying, dipping, dip-coating, melting, heating, sintering, welding, extrusion, injection molding, thermoforming, compression molding, blow molding, compaction, vapor deposition, knife coating, gravure coating, hot melt coating, silk screen coating, slot die coating, spin coating, and lithography.

The method of protecting an object against microbial infection, microbial colonization, or microbial transinfection can include, e.g., repelling, destroying, killing, and/or deactivating a microorganism. Because of the presence of the antimicrobial polymer in the antimicrobial substrate, the amount of microorganisms on a substrate and/or object is reduced compared to the amount of microorganisms on the same substrate and/or object, under the same conditions (e.g., temperature, relative humidity, light level, etc.), without the presence of the antimicrobial polymer. The level of reduction in the amount of microorganisms can be any level, including a 100% (e.g., 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, 20%, 15%, or 10%) reduction.

A “microorganism” (i.e., a microbe) as used herein can be a single cell or multicellular organism and includes organisms such as prokaryotes (e.g., bacteria and archaea), eukaryotes (e.g., protozoa, fungi, algae, microscopic plants and animals), and viruses. For example, the bacteria can be gram negative or gram positive. In specific embodiments, the microorganism is selected from Staphylococcus aureus, Streptococcus, Escherichia coli (E. coli), Pseudomonas aeruginosa, mycobacterium, adenovirus, rhinovirus, smallpox virus, influenza virus, herpes virus, human immunodeficiency virus (HIV), rabies, chikungunya, severe acute respiratory syndrome (SARS), polio, malaria, dengue fever, tuberculosis, meningitis, typhoid fever, yellow fever, ebola, shingella, listeria, yersinia, West Nile virus, protozoa, fungi Salmonella enterica, Candida albicans, Trichophyton mentagrophytes, poliovirus, Enterobacter aerogenes, Salmonella typhi, Klebsiella pneumonia, Aspergillus brasiliensis, and methicillin resistant Staphylococcus aureus (MRSA).

Without being bound by any particular theory, it is believed that the antimicrobial polymers described herein are effective by deactivating microbes, repelling microbes, or a combination of both deactivating and repelling microbes. For example, an antimicrobial polymer comprising one or more quaternary ammonium groups (e.g., a “polycation” polymer) can be coated onto a surface. A microbe can come into contact with the antimicrobial substrate and then be killed (e.g., cell membrane can rupture) due to the physical structure of the cation. It is believed that long hydrophobic alkyl substituents R¹, R², and/or R³ in the functionality —N⁺R¹R²R³ penetrate the membranes of the microbes with which it comes in contact. The penetration of the alkyl substituent(s) through the membrane of the microbe presumably leads to the rupturing of the membrane and killing of the microbe. This phenomenon has been compared to a “bubble-bursting porcupine” model. The killing or rupturing leads to the inactivation of the microbe.

In another example, it is believed that fluorinated groups part of an antimicrobial polymer provide a non-reactive and inert character to the substrate, hindering the attachment of microbes and their nutrients to the substrate. The inert character of highly fluorinated surfaces is well known in the literature as well as in commercial products (e.g., TEFLON™, fluorinated plastics in implants, and graft materials in surgical interventions). A microbe can diffuse onto or approach the substrate but will be repelled by the physical structure of the fluorinated group. If the microbe adheres to the substrate, its life is shortened due to an absence of nutrients.

An antimicrobial substrate comprising an antimicrobial polymer comprising at least one quaternary ammonium group and at least one fluorinated group can act via a combination of both mechanisms.

The substrate that is being modified can be of any suitable material, including a biocompatible material. The substrate can be used in or derived from any suitable form, such as, for example, a powder, dust, an aggregate, an amorphous solid, a sheet, a fiber, a tube, a fabric, or the like. In certain aspects, the substrate has a reactive functional group on its surface or the surface of the substrate can be modified to provide a reactive functional group capable of forming a covalent bond with the antimicrobial polymer. The functional group can be, for example, amino, ammonium, hydroxyl, mercapto, sulfone (e.g., —RSO₂R′), sulfinic acid (e.g., —RSO(OH)), sulfonic acid (e.g., —RSO₂(OH)), thiocyanate, thione, thial (e.g., —C(S)H or —RC(S)H), carboxyl, halocarboxy (e.g., —OC(O)X), halo, imido, anhydrido, alkenyl, alkynyl, phenyl, benzyl, carbonyl, formyl, haloformyl (e.g., —RC(O)X), carbonato, ester, alkoxy, phenoxy, hydroperoxy, peroxy, ether, glycidyl, epoxy, hemiacetal (e.g., —OCH(R)OH or —CH(OR)OH)), hemiketal (e.g., —OCRR′OH or —CR(OR′)OH), acetal (e.g., —OCHR(OR′) or —CH(OR)(OR′)), ketal (e.g., —OCRR′(OR″) or —CR(OR′)(OR″)), orthoester, orthocarbonate ester, amido (e.g., —C(O)NRR′ or —NRC(O)R′), imino, imido, azido, azo, cyano, nitrato, nitrilo, nitrito, nitro, nitroso, pyridinyl, phosphinyl, phosphonic acid, phosphate, phosphoester, phosphodiester, boronic acid, boronic ester, borinic acid, borinic ester, or a combination thereof. In the foregoing examples, R, R′, and R″ are H, alkyl, or cycloalkyl as described herein, and X is halo.

If an appropriate functional group is not present on the surface of the substrate, typically a suitable functional group can be provided by a chemical transformation. In general, a chemical transformation can be hydrolysis, oxidation (e.g., using Collins reagent, Dess-Martin periodinane, Jones reagent, and potassium permanganate), reduction (e.g., using sodium borohydride or lithium aluminum hydride), alkylation, deprotonation, electrophilic addition (e.g., halogenation, hydrohalogenation, hydration), hydrogenation, esterification, elimination reaction (e.g., dehydration), nucleophilic substitution, radical substitution, or a rearrangement reaction. If needed, more than one chemical transformation can be used to provide a suitable functional group for covalently bonding the antimicrobial polymer. Alternatively, a monomer with a desired functional group can be grafted to the substrate.

In some embodiments, the chemical transformation is hydrolysis. Generally, the hydrolysis is performed with water in the presence of a strong acid (e.g., strong inorganic acid, such as hydrochloric acid, sulfuric acid, phosphoric acid, nitric acid, hydroiodic acid, hydrobromic acid, chloric acid, and perchloric acid) or strong base (e.g., Group I and Group II hydroxides, such as lithium hydroxide, sodium hydroxide, potassium hydroxide, rubidium hydroxide, cesium hydroxide, magnesium hydroxide, calcium hydroxide, and barium hydroxide; ammonium hydroxide; and sodium carbonate). For example, a substrate comprising an acyl halide can undergo hydrolysis to form a carboxylic acid.

In some embodiments, the chemical transformation is a substitution reaction. For example, a substrate comprising a haloalkyl group can react with a strong base, as described herein, to form a hydroxy group.

In other aspects, the chemical transformation is alkylation, hydrogenation, or reduction. For example, a substrate comprising a hydroxy or haloalkyl (e.g., iodoalkyl or bromoalkyl) moiety can be reacted with ammonia to form an amino group. A substrate comprising a haloalkyl moiety also can be converted to a mercapto group by S-alkylation using thiourea. A substrate comprising a nitrile can be hydrogenated to form an amino group. A substrate comprising an amido group can be reduced (e.g., in the presence of lithium aluminum hydride) to form an amino group. A substrate comprising a formyl or keto group can be reduced to form an amino or hydroxy group.

In some embodiments, the reactive functional group is hydroxy, mercapto, or amino. In some aspects, the hydroxy, mercapto, or amino group is present on the surface of the substrate, whereas in other aspects, the hydroxy, mercapto, or amino group is formed on the surface by a chemical transformation. In an example, a substrate comprising an alkenyl group can undergo an acid catalyzed hydration reaction to form a secondary alcohol with a free hydroxy group.

To form a covalent bond, the antimicrobial polymer includes, or is modified to include, at least one functional group that will react with the functional group on the surface of the substrate. In some embodiments, the functional group for covalently bonding the substrate will be other than the quaternary ammonium or fluorinated group described above. The functional group on the polymer can be the same type of moiety described with respect to the surface of the substrate. As with the substrate, the functional group can either be present on the polymer or formed on the polymer via a chemical transformation as described herein for modifying the substrate.

The antimicrobial substrate can be formed by any suitable method using suitable temperatures (e.g., room temperature, reflux), reaction times, solvents, catalysts, and concentrations. In some aspects, an excess amount of antimicrobial polymer will be used to ensure an effective amount of polymer is bound to the substrate.

The antimicrobial polymer of the disclosure can be bound to the substrate in any suitable concentration. Typically, the concentration will be an amount effective to provide a desired result, e.g., provide an antimicrobial effect. Examples of concentrations of polymer include at least 0.001 part by weight polymer relative to 1 part by volume of carrier (e.g., at least 0.002 part by weight polymer, at least 0.005 part by weight polymer, at least 0.01 part by weight polymer, at least 0.015 part by weight polymer, at least 0.02 part by weight polymer, at least 0.025 part by weight polymer, at least 0.03 part by weight polymer, at least 0.04 part by weight polymer, at least 0.05 part by weight polymer, at least 0.06 part by weight polymer, at least 0.08 part by weight polymer, at least 0.1 part by weight polymer, at least 0.2 part by weight polymer, at least 0.3 part by weight polymer, at least 0.4 part by weight polymer, at least 0.5 part by weight polymer, at least 0.6 part by weight polymer, at least 0.8 part by weight polymer, and at least 1 part by weight polymer). The maximum amount of polymer is not particularly limited, but typically will be about 10 parts by weight polymer relative to 1 part by volume of carrier or less (e.g., about 9 parts by weight polymer or less, about 8 parts by weight polymer or less, about 7 parts by weight polymer or less, about 6 parts by weight polymer or less, about 5 parts by weight polymer or less, about 4 parts by weight polymer or less, about 3 parts by weight polymer or less, about 2 parts by weight polymer or less, about 1 parts by weight polymer or less, or about 0.5 parts by weight polymer or less).

If necessary, a linking group between the substrate and antimicrobial polymer can be present. In some embodiments, the linker is a bifunctional linker. Bifunctional linkers are known in the art (e.g., Sigma-Aldrich, St. Louis, Mo.). The bifunctional linker comprises any moiety that can form a chemical bond between the functional group on the substrate surface and at least one functional group on the polymer other than the quaternary ammonium group or fluorinated group. The linker can be of any suitable charge, length and/or rigidity. In some embodiments, the bifunctional linker is derived from a compound comprising one or more amino groups, hydroxyl, mercapto, halo groups, carboxyl groups, aryl, heteroaryl, or heterocyclyl groups prior to reaction with the substrate and/or antimicrobial polymer. Examples of a suitable heteroaryl include

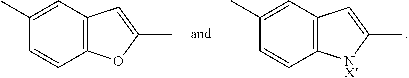

In some embodiments, these groups are at the terminal ends of the bifunctional linker. Examples of the linker include, e.g., bromobutyryl chloride, alkyne-PEG5-acid, amino-PEG4-alkyne, 2-(Boc-amino)ethanethiol, 4-(Boc-amino)-1-butanol, 4-(Boc-amino)butyl bromide, 4-bromobutyric acid, 6-bromo-1-hexanol, N-(3-bromopropyl)phthalimide, tert-butyl 4-hydroxybutyrate, N-(2-hydroxyethyl)trifluoroacetamide, N-(6-hydroxyhexyl)trifluoroacetamide, 4-mercapto-1-butanol, 6-mercapto-1-hexanol, phenacyl 4-(bromomethyl)phenylacetate, and diethylene glycol monoallyl ether.

Provided is a method of adjusting the hydrophobicity, hydrophilicity, and/or fluorophilicity of an antimicrobial substrate described herein. The method comprises selecting appropriate polymer components, including alkylene oxide and alkyl groups in the backbone and branches and their number of repeat units and density of branches, and selecting a quaternary ammonium group or groups and/or a fluorinated group or groups to provide a polymer-coated substrate that is hydrophobic, hydrophilic, and/or fluorophilic.

Also provided is a method of adjusting the hydrophobicity, hydrophilicity, fluorophilicity, and/or microbicidal performance of an antimicrobial substrate as described herein comprising selecting an appropriate concentration and method of attachment of the polymer to the substrate.

In embodiments, the substrate comprises natural polymer, synthetic polymer, fiber, wood, metal, ceramic, porcelain, stone, marble, cement, rubber, glass, silica, sand, or a combination thereof. In some aspects, the substrate comprises cotton, wool, nylon, polyester, metal, ceramic, porcelain, stone, marble, cement, rubber, or glass.

Metal substrates suitable for use in the disclosure include, for example, stainless steel, nickel, titanium, tantalum, aluminum, copper, gold, silver, platinum, zinc, Nitinol, Inconel, iridium, tungsten, silicon, magnesium, tin, alloys, coatings containing any of the foregoing, galvanized steel, hot dipped galvanized steel, electrogalvanized steel, annealed hot dipped galvanized steel, and combinations thereof

Glass substrates suitable for use in the disclosure include, for example, soda lime glass, strontium glass, borosilicate glass, barium glass, glass-ceramics containing lanthanum as well as combinations thereof

Silica substrates suitable for use in the disclosure include, for example, quartz, fused quartz, crystalline silica, fumed silica, silica gel, and silica aerogel.

Sand substrates suitable for use in the disclosure include, for example, sand comprised of silica (e.g., quartz), calcium carbonate (e.g., aragonite), and mixtures thereof. The sand can comprise other components, such as minerals (e.g., magnetite, chlorite, glauconite, gypsum, olivine, garnet), metal (e.g., iron), shells, coral, limestone, and rock.

Wood substrates suitable for the disclosure include, for example, hard wood and soft wood, and materials engineered from wood, wood chips, or fiber (e.g., plywood, oriented strand board, laminated veneer lumber, composites, strand lumber, chipboard, hardboard, medium density fiberboard). Types of wood include alder, birch, elm, maple, willow, walnut, cherry, oak, hickory, poplar, pine, fir, and combinations thereof

Fiber substrates suitable for use in the disclosure include, for example, natural fibers (e.g., derived from an animal, vegetable, or mineral) and synthetic fibers (e.g., derived from cellulose, mineral, or polymer). Suitable natural fibers include cotton, hemp, jute, flax, ramie, sisal, bagasse, wood fiber, silkworm silk, spider silk, sinew, catgut, wool, sea silk, wool, mohair, angora, and asbestos. Suitable synthetic fibers include rayon, modal, and Lyocell, metal fiber (e.g., copper, gold, silver, nickel, aluminum, iron), carbon fiber, silicon carbide fiber, bamboo fiber, seacell, nylon, polyester, polyvinyl chloride fiber (e.g., vinyon), polyolefin fiber (e.g., polyethylene, polypropylene), acrylic polyester fiber, aramid (e.g., TWARON™, KEVLAR™, or NOMEX™) and spandex.

Natural polymer substrates suitable for use in the disclosure include, for example, a polysaccharide (e.g., cotton, cellulose), shellac, amber, wool, silk, natural rubber, and a biopolymer (e.g., a protein, an extracellular matrix component, collagen).

Synthetic polymer substrates suitable for use in the disclosure include, for example, polyvinylpyrrolidone, acrylics, acrylonitrile-butadiene-styrene, polyacrylonitrile, acetals, polyphenylene oxides, polyimides, polystyrene, polypropylene, polyethylene, polytetrafluoroethylene, polyvinylidene fluoride, polyvinyl chloride, polyethylenimine, polyesters, polyethers, polyamide, polyorthoester, polyanhydride, polysulfone, polyether sulfone, polycaprolactone, polyhydroxy-butyrate valerate, polylactones, polyurethanes, polycarbonates, polyethylene terephthalate, as well as copolymers and combinations thereof

Typical rubber substrates suitable for use in the disclosure include, for example, silicones, fluorosilicones, nitrile rubbers, silicone rubbers, polyisoprenes, sulfur-cured rubbers, butadiene-acrylonitrile rubbers, isoprene-acrylonitrile rubbers, and the like.

Ceramic substrates suitable for use in the disclosure include, for example, boron nitrides, silicon nitrides, aluminas, silicas, combinations thereof, and the like.

Stone substrates suitable for use in the disclosure include, for example, granite, quartz, quartzite, limestone, dolostone, sandstone, marble, soapstone, and serpentine.

The antimicrobial substrate typically is a component of a larger structure or object. For example, the substrate can be part of a medical device, diagnostic equipment, implant, glove, mask, curtain, mattress, sheets, blankets, gauze, dressing, tissue, surgical drape, tubing, surgical instrument, safety gear, fabric, apparel item, floor, handles, wall, sink, shower or tub, or toilet. The antimicrobial substrate can also be part of furniture, a wall switch, toy, athletic equipment, playground equipment, shopping cart, countertop, appliance, railing, door, air filter, air processing equipment, water filter, water processing equipment, pipe, phone, cell phone, remote control, computer, mouse, keyboard, touch screen, leather, cosmetic, cosmetic making equipment, cosmetic storage equipment, personal care item, personal care item making equipment, personal care storage equipment, animal care item, animal care item making equipment, animal care storage equipment, veterinary equipment, powder, cream, gel, salve, eye care item, eye care item making equipment, eye care storage equipment, contact lens, contact lens case, glasses, jewelry, jewelry making equipment, or jewelry storage equipment. The antimicrobial substrate can further be part of a utensil, dish, cup, container, object display container, food display container, food package, food processing equipment, food handling equipment, food transportation equipment, food storage equipment, or food vending equipment. The antimicrobial substrate can further be part of animal housing, farming equipment, animal food handling equipment, animal food storage space, animal processing equipment, animal food storage equipment, or animal food container. The antimicrobial substrate can further be part of an air vehicle, land vehicle, water vehicle, water storage space, water processing equipment, water storage equipment, water storage container, or water filter.

A “medical device” includes any device having surfaces that contact tissue, blood, or other bodily fluids in the course of their use or operation, which are found on or are subsequently used within a mammal (e.g., a human). Medical devices include, for example, extracorporeal devices for use in surgery, such as blood oxygenators, blood pumps, blood storage bags, blood collection tubes, blood filters including filtration media, dialysis membranes, tubing used to carry blood and the like which contact blood which is then returned to the patient or mammal Medical devices also include endoprostheses implanted in a mammal (e.g., a human), such as vascular grafts, stents, pacemaker leads, surgical prosthetic conduits, heart valves, and the like, that are implanted in blood vessels or the heart. Medical devices also include devices for temporary intravascular use such as catheters, guide wires, amniocentesis and biopsy needles, cannulae, drainage tubes, shunts, sensors, transducers, probes and the like which are placed into the blood vessels, the heart, organs or tissues for purposes of monitoring or repair or treatment. Medical devices also include prostheses such as artificial joints such as hips or knees as well as artificial hearts. In addition, medical devices include penile implants, condoms, tampons, sanitary napkins, ocular lenses, sling materials, sutures, hemostats used in surgery, antimicrobial materials, surgical mesh, transdermal patches, and wound dressings/bandages.

The “diagnostic equipment” includes any device or tool used to diagnose or monitor a medical condition. Examples include an ultrasound, MRI machine, PET scanner, CT scanner, ventilator, heart-lung machine, ECMO machine, dialysis machine, blood pressure monitor, otoscope, ophthalmoscope, stethoscope, sphygmomanometer, blood pressure cuff, electrocardiograph, thermometer, defibrillator, speculum, sigmoidoscope, and anoscope.

The “surgical instrument” includes any tool or device used for performing surgery or an operation. Examples include a scalpel, lancet, trocar, hemostat, grasper, forceps, clamp, retactor, distractor, positioner, tracheotome, dilator, stapler, irrigation needle, injection needle, drill, scope, endoscope, probe, ruler, and caliper.

“Safety gear” includes devices used to protect a person, animal, or object. Examples of “safety gear” are a mask, face shield, visor, goggles, glasses, gloves, shoe covers, foot guard, leg guard, belt, smock, apron, coat, vest, raingear, hat, helmet, chin strap, hairnet, shower cap, hearing protection (ear plugs, ear muffins, hearing bands), respirator, gas mask, supplied air hood, collar, leash, and first aid kit.

“Fabric” includes any type of suitable fabric, such as bedding, curtains, towels, table coverings, protective sheeting, and dish cloths.

An “apparel item” includes an item of clothing, footwear, or other item someone would wear on his/her person. Examples include a uniform, coat, shirt, pants, waders, scrubs, gowns, aprons, socks, shoe or boot liner, an insole, gloves, hats, shoes, boots, and sandals.

The antimicrobial substrate can be part of a building structure or an item that can be found in a building structure, such as a floor, wall, an appliance (e.g., a refrigerator, oven, stove, dishwasher, washing machine, clothes dryer, furnace, water heater, air conditioner, heater), sink, shower or tub, toilet, furniture (e.g., mattress, couch, sofa, chair, table, shelf, mantle, bed, dresser), countertop, railing, air filter, air processing equipment, water processing equipment, water filter, pipe, or door.

The antimicrobial substrate can also be a toy or athletic equipment, including exercise equipment, playground equipment, or a pool.

The antimicrobial substrate can be a utensil (e.g., knife, fork, spoon, ladle, spatula, whisk, etc.), a dish (e.g., a food storage container, a food serving piece, etc.), a food package (e.g., a bag, a box, foil, plastic wrap), or other item that comes in contact with food (e.g., a cutting board, food display container, food processing equipment, food handling equipment, food transportation equipment, food storage equipment, food vending equipment, animal food handling equipment, animal food processing equipment, animal food storage equipment, animal food storage space, animal food container). The antimicrobial substrate can be part of food processing equipment, such as food processing tanks, stirrers, conveyor belts, knives, grinders, packaging machines, labeling machines, etc.

The antimicrobial substrate can be part of an electronic device, such as a phone, cell phone, remote control, computer, mouse, keyboard, and touch screen.

The antimicrobial substrate can further be part of a cosmetic (e.g., eye shadow, eyeliner, primer, foundation, lipstick, lip gloss, blush), cosmetic making equipment, cosmetic storage equipment, cosmetic packaging equipment, a personal care item (e.g., cream, gel, salve, lip balm, body soap, facial soap, lotion, cologne, perfume, antiperspirant, deodorant, facial tissue, cotton swabs, cotton pads, mouthwash, toothpaste, nail polish, shampoo, conditioner, hairspray, talcum powder, shaving cream, contact lens, contact lens case, glasses), personal care item making equipment, personal care storage equipment, personal care packaging equipment, jewelry (e.g., necklace, ring, earring, bracelet, watch), jewelry making equipment, or jewelry storage equipment.

The “animal care item” and “veterinary equipment” can be any product used in a setting that includes animals, such as a house, boarding house, or veterinary hospital. Of course, veterinary equipment can be used at a location outside of a hospital setting. Animals are any animals that are typically considered pets, non-pets, boarded, treated by a veterinarian, and animals in the wild. Examples include a dog, cat, reptile, bird, rabbit, ferret, guinea pig, hamster, rat, mouse, fish, turtle, horse, goat, cattle, and pigs. Suitable animal care items include the personal care items described herein, toys, bed, crate, kennel, carrier, bowl, dish, leash, collar, litterbox, and grooming items (e.g., clippers, scissors, a brush, comb, dematting tool, and deshedding tool). Suitable veterinary equipment includes any of the medical devices and surgical instruments described herein and other equipment, such as a table, tub, stretcher, sink, scale, cage, carrier, and leash.

The “animal housing” can be any suitable housing, such as a coop, stable, shelter, grab bag shelter, hutch, barn, shed, pen, nestbox, feeder, stanchion, cage, carrier, or bed.

The “farming equipment” is any device used in an agricultural setting, including a farm or ranch, particularly a farm or ranch that houses animals, processes animals, or both. Animal livestock that can be housed or processed as described herein and include, e.g., horses, cattle, bison, and small animals such as poultry (e.g., chickens, quails, turkeys, geese, ducks, pigeons, doves, pheasants, swan, ostrich, guineafowl, Indian peafowl, emu), pigs, sheep, goats, alpacas, llamas, deer, donkeys, rabbits, and fish. Examples of farming equipment include as a wagon, trailer, cart, barn, shed, fencing, sprinkler, shovel, scraper, halter, rope, restraining equipment, feeder, waterer, trough, water filter, water processing equipment, stock tank, fountain, bucket, pail, hay rack, scale, poultry flooring, egg handling equipment, a barn curtain, tractor, seeder, planter, plow, rotator, tiller, spreader, sprayer, agitator, sorter, baler, harvester, cotton picker, thresher, mower, backhoe loader, squeeze chute, hydraulic chute, head chute, head gate, crowding tub, corral tub, alley, calving pen, calf table, and milking machine.

The antimicrobial substrate can be part of a vehicle, such as an air vehicle, land vehicle, or water vehicle. Suitable vehicles include a car, van, truck, bus, ambulance, recreational vehicle, camper, motorcycle, scooter, bicycle, wheelchair, train, streetcar, ship, boat, canoe, submarine, an unmanned underwater vehicle (UUV), a personal water craft, airplane, jet, helicopter, unmanned autonomous vehicle (UAV), and hot air balloon.

The following examples further illustrate the disclosure but, of course, should not be construed as in any way limiting its scope.

### EXAMPLES

### Example 1

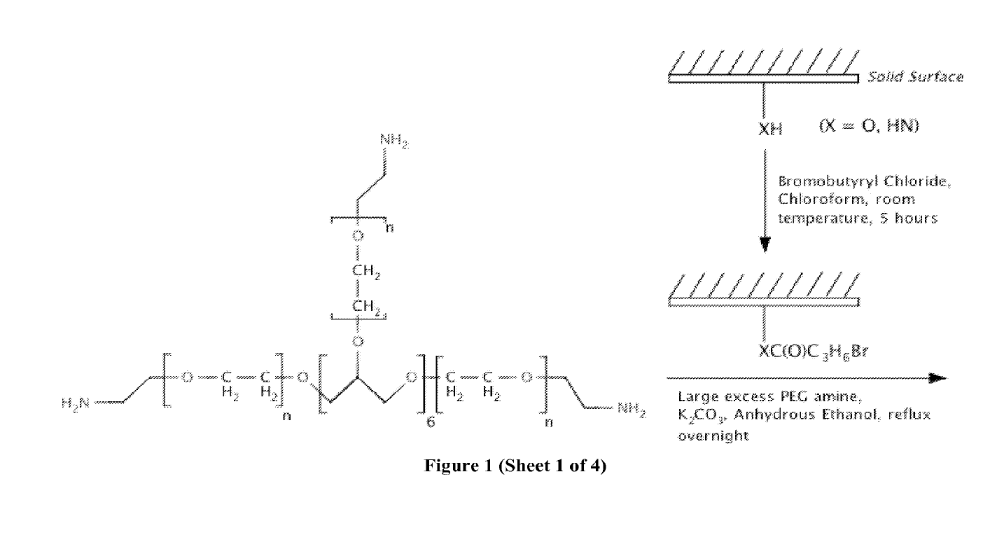

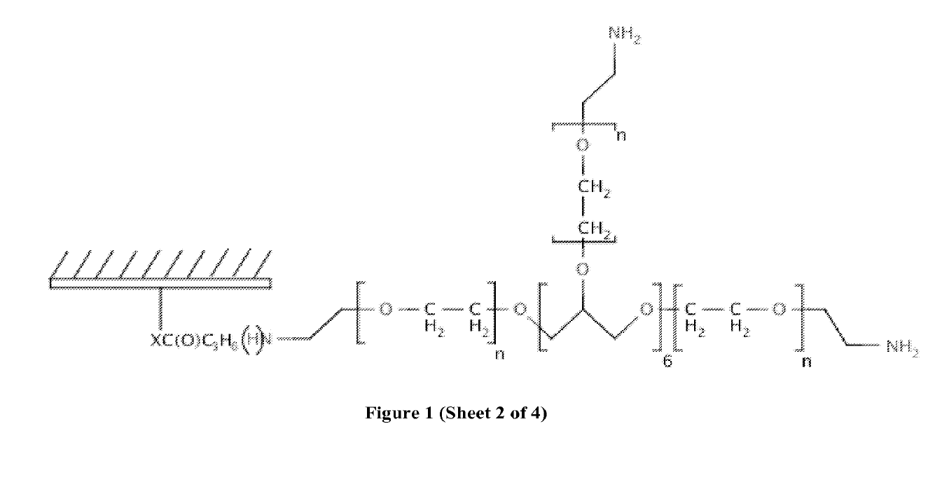

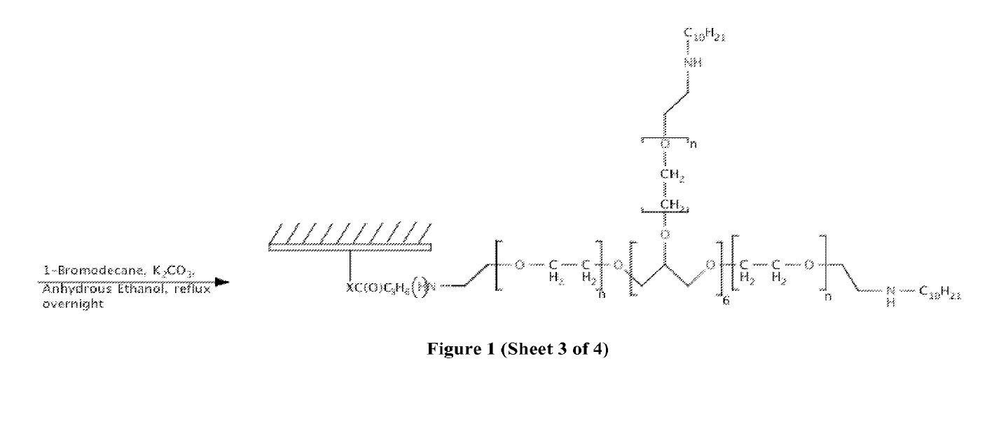

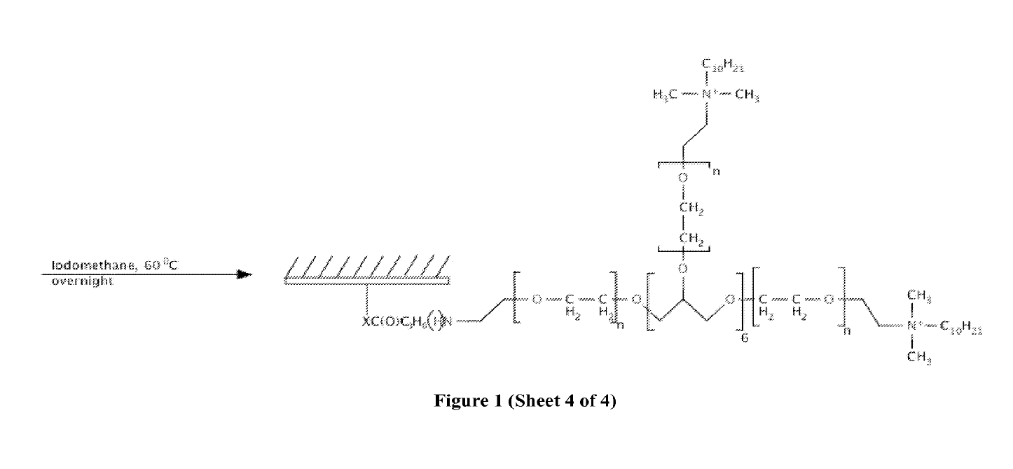

This example illustrates a method of preparing an antimicrobial substrate in accordance with an embodiment of the disclosure. A 25 cm² cloth of cotton is allowed to stir in a 100 mL solution of chloroform containing 5 mL (43.1 mmol) of 4-bromobutyryl chloride for 5 hours at room temperature. The resulting acylated cloth is washed with chloroform to remove unreacted butyryl chloride before stirring it in a solution of large excess of dry amine terminated PEG (0.733 g, 0.0733 mmol, 0.586 mmol eq. of NH₂) (FIG. 1) and 0.8104 g, 10 eq., 5.86 mmol of potassium carbonate in 100 ml of anhydrous ethanol at its refluxing temperature overnight. The resulting cloth is rinsed with ethanol to remove unreacted reagents before re-submerging it in 100 mL dry ethanol containing 1.216 mL, 10 eq., 5.86 mmol of 1-bromodecane and 5.86 mmol of potassium carbonate. The mixture is brought to reflux overnight.

On the following day, the cloth is once more rinsed with ethanol and re-submerged in 100 mL dry ethanol containing 4.159 g, 50 eq., 29.3 mmol iodomethane, as 14.65 mL of a 2 M solution in t-butyl methyl ether, and the resulting mixture is further stirred at 60° C. overnight. The resulting cloth is rinsed ethanol, hexanes, then water in order to remove all unreacted chemicals. The resulting product is cotton functionalized with polycation N,N-decyl, methyl-PEG (10k, ca. 7-arm).

### Example 2

To test the antimicrobial effect of an antimicrobial substrate of the present disclosure, the following method can be used.

Preparation of the Escherichia coli (E. coli) Inoculum

Lysogeny broth (LB) (1 mL) is added to a 15 ml cell culture tube. Seven (7) μl of 40% glycerol stock of the ONE SHOT™ TOP10 E. coli cells (Life Technologies, Grand Island, N.Y.) (from the −20° C. freezer) is inoculated into the 1 ml LB broth. The inoculate is incubated at 37° C. for 3 hours with constant shaking (ca. 250 rpm). The inoculate is then centrifuged at 2500 rpm speed for 4 min. The LB broth is separated, and the E. coli cells are washed twice with 1 ml of sterile phosphate buffered saline (PBS) 1× (pH 7.4) each time and then re-suspended in 1 mL of the same buffer. Using optical density measurements, the final concentration is estimated to be 10⁷-10⁸ cells/ml.

### Preparing LB Agar Medium (1 L):

In a 1 L French style bottle equipped with a magnetic stirrer is added 800 ml dH₂O (deionized MILLI-Q™ water), in which 10 g tryptone (BD 211705, pancreatic digest of casein), 5 g yeast extract (BD 212750, extract from autolysed yeast cells), 5 g NaCl (Sigma-Aldrich, St. Louis, Mo.) is dissolved. The pH is checked and adjusted to 7.0 with a concentrated NaOH (1-5 M), if necessary. dH₂O is added to provide a final volume of 1000 ml. Agar powder (15 g) (final concentration=1.5%) (BD 214530, DIFCO™ Agar Granulated) is added. The mixture is stirred to provide the complete dissolution (or at least suspension) of agar into solution. The mixture is sterilized by autoclaving at 15 psi and 121-124° C. for 15-25 minutes. The resulting mixture is cooled to 45° C. in a water bath.

Applying E. coli Inoculum to Treated and Untreated Cotton:

About 100 μL of the E. coli solution in PBS is spread over each slide with a plastic pipette tip. The solution is allowed to dry for 5 min in air. The E. coli solution is covered with an LB agar layer by pouring the 45° C. warm solution on top of (i) a 25 cm² untreated cotton cloth and (ii) the 25 cm² treated cotton cloth prepared in Example 1. The cotton samples were allowed to dry inside a biosafety cabinet for 30 min, and then incubated at 37° C. overnight.

On the following day, the treated cotton cloth from Example 1 will have limited-to-no bacterial growth relative to the untreated cotton cloth (control).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the disclosure (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein. Variations of those embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. An antimicrobial substrate comprising a substrate to which is covalently bonded an antimicrobial polymer,
wherein said antimicrobial polymer comprises an alkylene oxide backbone to which are attached one or more alkyl and/or alkylene oxide primary branches,
wherein at least one of the alkyl or alkylene oxide primary branches is functionalized with a quaternary ammonium group or a fluorinated group, or at least two of the alkyl and/or alkylene oxide primary branches are functionalized with a quaternary ammonium group and a fluorinated group,
wherein at least one of the alkyl and/or alkylene oxide primary branches optionally contains one or more alkyl and/or alkylene oxide secondary branches that are functionalized with a quaternary ammonium group or a fluorinated group, or at least two of the alkyl and/or alkylene oxide secondary branches are functionalized with a quaternary ammonium group and a fluorinated group, and
wherein said polymer is associated with an anion to maintain electro-neutrality when a quaternary ammonium group is present.

2. The antimicrobial substrate of claim 1, wherein the alkylene oxide backbone of the antimicrobial polymer comprises a propylene oxide backbone, an ethylene oxide backbone, or a backbone comprising both propylene oxide and ethylene oxide units.

3. The antimicrobial substrate of claim 1, wherein the alkylene oxide primary branch comprises at least one ethylene oxide unit or at least one propylene oxide unit.

4. The antimicrobial substrate of claim 1, wherein the quaternary ammonium or fluorinated group is at a terminus of at least one of the alkyl or alkylene oxide primary branches.

5. The antimicrobial substrate of claim 1, wherein the alkylene oxide backbone is attached to at least 2 to 20 alkyl and/or alkylene oxide primary branches.

6. (canceled)

7. The antimicrobial substrate of claim 2, wherein the propylene oxide backbone has a formula: 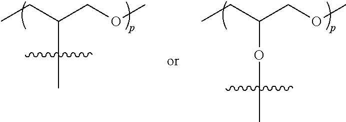
wherein p is 1 to 60.

8. The antimicrobial substrate of claim 1, wherein the alkyl and/or alkylene oxide primary branch, or the alkyl and/or alkylene oxide secondary branch, that is functionalized has of the formula
—(CH₂)ₙ—X,—(CH₂CH₂O)ₙ—X, or —(CH₂CH₂CH₂O)ₙ—X,
wherein:
X is —(CH₂)ₘ—Y, when Y is a quaternary ammonium group or
X is —(CH₂)ₘ—NHC(O)—Y, when Y is a fluorinated group,
m is 0 to 10, and
n is 1 to 2,500.

9. The antimicrobial substrate of claim 1, wherein at least one of the alkyl or alkylene oxide primary branches is functionalized with a quaternary ammonium group.

10. The antimicrobial substrate of claim 1, wherein the quaternary ammonium group has the formula —N⁺R¹R²R³, wherein R¹, R², and R³ are independently selected from alkyl, alkenyl, cycloalkyl, and aryl.

11-14. (canceled)

15. The antimicrobial substrate of claim 1, wherein at least one of the alkyl or alkylene oxide primary branches is functionalized with a fluorinated group.

16-19. (canceled)

20. The antimicrobial substrate of claim 1, wherein at least two of the alkyl and/or alkylene oxide primary branches are functionalized with a quaternary ammonium group and a fluorinated group.

21. The antimicrobial substrate of claim 1, wherein the substrate has a reactive functional group on its surface or the surface of the substrate can be modified to provide a reactive functional group capable of forming a covalent bond.

22. The antimicrobial substrate of claim 21, wherein the reactive functional group is amino, ammonium, hydroxyl, mercapto, sulfone, sulfinic acid, sulfonic acid, thiocyanate, thione, thial, carboxyl, halocarboxy, halo, imido, anhydrido, alkenyl, alkynyl, phenyl, benzyl, carbonyl, formyl, haloformyl, carbonato, ester, alkoxy, phenoxy, hydroperoxy, peroxy, ether, glycidyl, epoxy, hemiacetal, hemiketal, acetal, ketal, orthoester, orthocarbonate ester, amido, imino, imido, azido, azo, cyano, nitrato, nitrilo, nitrito, nitro, nitroso, pyridinyl, phosphinyl, phosphonic acid, phosphate, phosphoester, phosphodiester, boronic acid, boronic ester, borinic acid, borinic ester, or a combination thereof.

23-25. (canceled)

26. The antimicrobial substrate of claim 21, wherein the substrate is natural polymer, synthetic polymer, fiber, wood, metal, ceramic, porcelain, stone, marble, cement, rubber, glass, silica, or sand.

27. The antimicrobial substrate of claim 1, which is 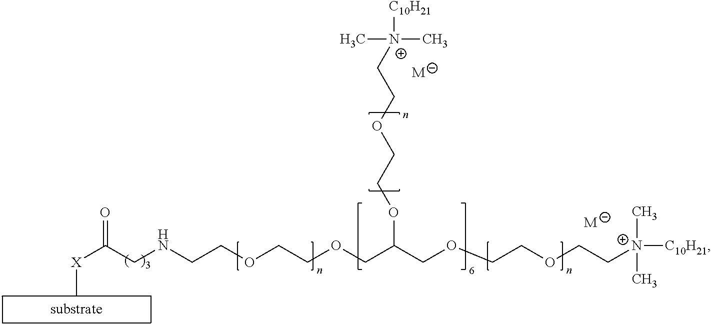
wherein:
X is O, S, or NH;
M⁻ is an anion; and
each n is independently 2 to 2500.

28. The antimicrobial substrate of claim 1, wherein the antimicrobial polymer has a polycation selected from: polycation N,N-octadecyl, methyl-PEG; polycation N,N-dodecyl, methyl-PEG; polycation N,N-decyl, methyl-PEG; polycation N,N-octyl, methyl-PEG; polycation N,N-hexyl, methyl-PEG; and polycation N,N-hexyl, methyl-PEG.

29. The antimicrobial substrate of claim 1, wherein the antimicrobial polymer has a polycation selected from: polycation N,N-octadecyl, methyl-PEG(440, ca. 2-arm); polycation N,N-dodecyl, methyl-PEG(440, ca. 2-arm); polycation N,N-octadecyl, methyl-PEG(2k, ca. 3-arm); polycation N,N-dodecyl, methyl-PEG(2k, ca. 3-arm); polycation N,N-octadecyl, methyl-PEG(3k, ca. 3-arm); polycation N,N-dodecyl, methyl-PEG(3k, ca. 3-arm); polycation N,N-decyl, methyl-PEG(10k, ca. 7-arm); polycation N,N-decyl, methyl-PEG(10k, ca. 3-arm); polycation N,N-decyl, methyl-PEG(20k, ca. 7-arm); polycation N,N-decyl, methyl-PEG(20k, ca. 3-arm); polycation N,N-decyl, methyl-PEG(40k, ca. 7-arm); polycation N,N-decyl, methyl-PEG(40k, ca. 3-arm); polycation N,N-octyl, methyl-PEG(10k, ca. 7-arm); polycation N,N-octyl, methyl-PEG(10k, ca. 3-arm); polycation N,N-octyl, methyl-PEG(20k, ca. 7-arm); polycation N,N-octyl, methyl-PEG(20k, ca. 3-arm); polycation N,N-octyl, methyl-PEG(40k, ca. 7-arm); polycation N,N-octyl, methyl-PEG(40k, ca. 3-arm); polycation N,N-hexyl, methyl-PEG(10k, ca. 7-arm); polycation N,N-hexyl, methyl-PEG(10k, ca. 3-arm); polycation N,N-hexyl, methyl-PEG(20k, ca. 7-arm); polycation N,N-hexyl, methyl-PEG(20k, ca. 3-arm); polycation N,N-hexyl, methyl-PEG(40k, ca. 7-arm); and polycation N,N-hexyl, methyl-PEG(40k, ca. 3-arm).

30-34. (canceled)

35. A method for protecting an object against microbial infection, microbial colonization, or microbial transinfection comprising providing to the object an antimicrobial substrate according to claim 1.

36-37. (canceled)

38. A method of adjusting the hydrophobicity, hydrophilicity, and/or fluorophilicity of the antimicrobial substrate of claim 1 comprising selecting appropriate polymer components, including alkylene oxide and alkyl groups in the backbone and branches and their number of repeat units and density of branches, and selecting a quaternary ammonium group or groups and/or a fluorinated group or groups to provide a polymer-coated substrate that is hydrophobic, hydrophilic, and/or fluorophilic.

39. A method of adjusting the hydrophobicity, hydrophilicity, fluorophilicity, and/or microbicidal performance of the antimicrobial substrate of claim 1 comprising selecting an appropriate concentration and method of attachment of the polymer to the substrate.
